(19) United States
(12) Patent Application Publication
(10) Pub. No.: US 2018/0291387 A1
(43) Pub. Date: Oct. 11, 2018

(54) ENHANCED PLANTS OF GENUS HUMULUS AND METHODS OF MAKING AND USING THE SAME

(71) Applicant: ebbu, LLC, Evergreen, CO (US)

(72) Inventor: Robert Fletcher ROSCOW, JR., Boulder, CO (US)

(21) Appl. No.: 15/950,713

(22) Filed: Apr. 11, 2018

Related U.S. Application Data
(60) Provisional application No. 62/484,373, filed on Apr. 11, 2017.

(51) Int. Cl. C12N 15/82

(52) U.S. Cl. CPC C12N 15/8223; C12N 15/8243

(57) ABSTRACT

Disclosed herein is a genetically modified plant of genus humulus not requiring flowering for producing lupulin glands comprising secondary compounds. Provided herein is a new plant of genus humulus having lupulin glands on non-flowering parts of the plant, such as leaves. The disclosed plants have a high mass % of secondary compounds and a high degree of lupulin glands coverage on the surface of the plant. Also disclosed herein are methods of producing secondary compounds from a plant of genus humulus without flowering the plant of genus humulus. For example, the disclosed methods provide for inducing lupulin glands development on a plant of genus humulus without flowering the plant of genus humulus.

### CROSS REFERENCE TO OTHER RELATED APPLICATIONS

This application claims benefit under 35 U.S.C. 119(e) of U.S. Provisional Application No. 62/484,373 filed Apr. 11, 2017, which is hereby incorporated by reference in its entirety.

### TECHNICAL FIELD

This disclosure relates to the hops industry. In particular, this disclosure relates to cultivating plants of genus humulus comprising more lupulin glands.

### BACKGROUND

Hops are the flowers of the Humulus Lupulus plant. For centuries hops have been used in drinks and medicine. For example, hops are a primary ingredient in beer, imparting a bitter taste, sometimes described as “hoppy”. Volatile secondary compounds produced in the lupulin glands of the hop flower provide a flavor profile, e.g., citrus, herbal, fruit, etc.

Some examples of secondary compounds are alpha acids, beta acids, essential oils, flavonoids, ketones, aldehydes, hydrocarbons, alcohols, acids, epoxides, esters, sulphur compounds, and terpenes.

Only the female plant of genus humulus grows flowers. Pollination by male plants could produce seeds, which are undesirable for beer making. Plants of genus humulus grow by using bines as opposed to vines. A vine uses tendrils or suckers to climb a structure upward while a bine wraps around a structure in a helical shape and grows upwards. Cultivators hand train plants of genus humulus to grow on either twine or trellises in order to grow and produce hops. If a plant of genus humulus does not grab onto a structure the plant can fall back to the ground and not produce as many flowers. To effectively produce flowers, the soil should have a pH of 6-8 and be rich in potassium, phosphate, and nitrogen.

Cultivating plants of genus humulus requires dedicating large areas of space and building an infrastructure for cultivating hops. The initial harvest is not plentiful because in the first year. A majority of the energy and resources within the plant is dedicated to developing a root system. Once the root system is established subsequent harvests may produce more plentiful crops. Because the secondary compounds are found primarily in the lupulin glands, much of the plant goes to waste. If the crop does not produce a plentiful harvest the enterprise may fail to generate a reasonable return on their investment.

There exists a need for more efficient hop cultivation and secondary compound production. Accordingly, having plants of genus humulus with more lupulin glands on both the hops and plant itself would be provide significant benefits.

There exists a need for a plant of the genus humulus not requiring flowering in order to produce lupulin glands comprising secondary compounds. There exists a need for a plant of the genus humulus having lupulin glands on non-flowering parts of the plant, such as leaves. There exists a need for a plant of the genus humulus comprising a high mass % of secondary compounds. There exists a need for methods of producing secondary compounds from a plant of genus humulus without flowering the plant of genus humulus. There exists a need for methods of inducing lupulin glands development on a plant of genus humulus without flowering the plant of genus humulus.

### DETAILED DESCRIPTION

Disclosed herein are new plants of genus humulus. In one embodiment, the plants disclosed herein produce secondary compounds more efficiently than other plants of genus humulus. In one embodiment, the plants disclosed herein do not require flowering in order to produce lupulin glands. In one embodiment, the plant disclosed herein comprise lupulin glands on non-flowering parts of the plant, such as leaves. In one embodiment, the plants disclosed comprise a high mass % of secondary compounds.

Disclosed herein are new methods of producing secondary compounds from a plant of genus humulus without flowering. In one embodiment, the methods disclosed herein comprise inducing lupulin gland development on a plant of genus humulus without flowering the plant of genus humulus. In one embodiment, the methods disclosed herein comprise genetically modifying a plant of genus cannabis. In one embodiment, the methods disclosed herein comprise altering the genetic information of a plant of genus humulus. In one embodiment, the methods disclosed herein comprise adding genetic information from a non-native plant.

As used herein, the term “plant” means a multicellular eukaryote of the kingdom Plantae, whether naturally occurring, completely manmade, or some combination thereof.

As used herein, the term “plant of the genus humulus” means a plant belonging to the genus humulus within the accepted biological taxonomical system. In one embodiment, the plant of the genus humulus is Humulus lupulus.

Within the context of this disclosure, the term “hop” can refer to either a plant of genus humulus or a flower from a plant of genus humulus.

As used herein, the term “non-flowering part of the plant” refers to an area of the plant where reproductive structures do not normally develop. In one embodiment, the non-flowering part of a plant is a leaf. In one embodiment, the non-flowering part of a plant is the stem. In one embodiment, the non-flowering part of the plant refers to an area of the plant where lupulin glands do not commonly develop. For example, lupulin glands are commonly found within the leaves of the hop flower or cone. Within the context of this disclosure, lupulin glands may develop on the stem, outer leaves, roots, etc.

Disclosed herein is a plant of genus humulus, comprising;
lupulin glands on non-flowering parts of the plant of genus humulus;
a vector comprising genetic information; and
wherein the plant of genus humulus comprises a surface area.

In one embodiment, a vector is a plasmid. In one embodiment, a plasmid comprises a cDNA sequence native to a plant of genus humulus corresponding to lupulin gland induction.

In one embodiment, a vector is a bacteria or bacterium. In one embodiment, a bacteria is a purified transformed bacteria. In one embodiment, a purified transformed bacteria comprises a DNA sequence native to a plant of genus humulus, wherein said native DNA sequence corresponds to lupulin gland induction, wherein the plant of genus humulus comprises a surface area.

In one embodiment, the plants disclosed herein comprise two or more vectors. In one embodiment, a plant comprises two or more plasmids. In one embodiment, a plant comprises two or more purified transformed bacterias. In one embodiment, a plant comprises one or more vectors and one or more purified transformed bacterias. In one embodiment, the plants disclosed herein comprise two or more vectors comprising the same genetic information, e.g., the same DNA sequence. In one embodiment, the plants disclosed herein comprise two or more vectors comprising different genetic information, e.g., two or more different DNA sequences.

In one embodiment, the plants disclosed herein comprise a plasmid, wherein the plasmid comprises a cDNA sequence native to a plant of genus humulus corresponding to lupulin gland induction; and a purified transformed bacteria, wherein the purified transformed bacteria comprises a DNA sequence native to a plant of genus humulus, wherein said native DNA sequence corresponds to lupulin gland induction.

As used herein, the term “vector” refers to a vehicle (e.g., a plasmid, a chromosome, a cosmid, a bacteria, a virus, etc.) for delivering foreign material into an organism. In one embodiment, a vector comprises genetic information, e.g., a DNA sequence, a RNA sequence, etc. In one embodiment, a vector replicates autonomously. In some instances, a vector is also referred to as a “carrier.” In one embodiment, a vector is a unicellular organism, e.g., a yeast cell. In one embodiment, a vector is a chromosome, both natural and artificial.

As used herein, the term “plasmid” refers to a genetic structure capable of replicating independent of the chromosomes of an organism. In one embodiment, a plasmid comprises a double stranded circle of genetic information, e.g., a DNA strand. In one embodiment, a plasmid is used to introduce genetic information into an organism. In one embodiment, a plasmid is held within a cell, e.g., a bacterium, a vector, etc. In one embodiment, a plasmid comprises a pRI-201AN sequence. In one embodiment, a pRI-201AN comprises a set of useful genetic features for working with hop plants. In one embodiment, each part of the pRI-201AN sequence is individually replaced with alternative sequences having similar or slightly different functions.

As used herein, the term “cDNA” refers to complementary DNA, which is double-stranded DNA synthesized from a messenger RNA (mRNA) template in a reaction typically catalysed by the enzyme reverse transcriptase. In one embodiment, a cDNA strand and/or sequence comprises only the relevant genetic information of DNA, i.e., the cDNA strand comprises no introns. In one embodiment, a cDNA sequence comprises a genetic sequence for a protein. In one embodiment, a cDNA sequence comprises a genetic sequence for an enzyme.

Within the context of this disclosure, the term “cDNA” may refer to a naturally occurring, modified, or synthetic cDNA sequence and/or strand or combinations thereof in any proportion.

Within the context of this disclosure, a cDNA sequence native to a plant of genus humulus refers to a cDNA sequence resembling a naturally occurring DNA sequence found within a plant of genus humulus. In one embodiment, a cDNA sequence is sequence from a DNA sequence found within a plant of genus humulus. In one embodiment, a cDNA sequence is synthesized to resemble a DNA sequence from a plant of genus humulus.

In one embodiment, a cDNA sequence is validated.

Within the context of this disclosure, the term “validated” refers to ensuring the efficacy of genetic material. In one embodiment, a cDNA sequence is confirmed to have the same efficacy of a native strand of DNA via a technique such as restriction enzyme digest or Sanger sequencing.

As used herein, the term “lupulin gland induction” refers to the development of an organ comprising secondary compounds. Within the context of this disclosure, the term “lupulin gland induction” may refer to promoting biological processes for making or growing lupulin glands, such as synthesizing proteins for structural development. The term “lupulin gland induction” may also refer to thwarting biological processes decreasing lupulin gland production, such as interfering with a repressor of lupulin gland development. In one embodiment, a genetic sequence codes for a molecule contributing to lupulin gland development, e.g., a protein, an enzyme, a secondary compound, etc. In one embodiment, lupulin gland induction occurs as a result of another process, e.g., increasing the growth of a plant of genus humulus increases the development of lupulin glands.

As used herein, the term “lupulin gland” refers to an organ found on a plant of genus humulus. In one embodiment, a lupulin gland is epidermal outgrowth, protrusion, structure, and/or appendage. In one embodiment, a lupulin gland is a sac. In one embodiment, a lupulin gland is a hair. In one embodiment, a lupulin gland is a gland. In one embodiment, a lupulin gland is not exposed to the surrounding environment, e.g., a lupulin gland covered by bracts. Within the context of this disclosure, a lupulin gland may also be refer to a trichome. A trichome commonly refers to “hair-like” structure on a plant but may refer to another structure of a plant. In one embodiment, a trichome refers to a gland. In one embodiment, a trichome refers to a sac. In one embodiment, a trichome refers to a hair.

In one embodiment, lupulin glands are developed on the stem of a plant of genus humulus. In one embodiment, lupulin glands are developed on the leaf of a plant of genus humulus. In one embodiment, lupulin glands are developed on the bines of a hop plant. In one embodiment, lupulin glands are developed on the strig of a hop plant. In one embodiment, lupulin glands are developed on the bracteoles of a hop plant.

As used herein, the term “transformed bacteria” refers to a microorganism or microorganisms comprising non-native genetic material. In one embodiment, transformed bacteria introduces genetic information into another organism. For example, transformed bacteria is introduced into an organism and the organism absorbs the genetic information from the transformed bacteria. In one embodiment, transformed bacteria is a modified microorganism. For example, a DNA sequence is inserted into a bacterium of Agrobacterium tumifaciens. This genetically modified Agrobacterium tumifaciens bacteria is an example of a vector that is useful within the context of this disclosure for introducing genetic information into an organism or organisms. Other vectors could be used as alternatives to Agrobacterium tumifaciens bacteria without departing from the scope of this disclosure. Examples include, but are not limited, to biolistic transformation, protoplast transformation, etc.

Within the context of this disclosure, the term “purified transformed bacteria” refers to transformed bacteria which has been isolated from its natural surroundings. For example, a bacterium is harvested from a growth medium by centrifugation.

As used herein, the term “surface area” refers to the amount of space on the outermost layer of an object. In one embodiment, the “surface area” of an object can be calculated for various degrees of precision or accuracy. For example, the surface area of a plant is divided into small segments and the area of each segment is calculated and added together. The size of each segment is a factor in the accuracy of the final measurement. The shape of the plant may also be a factor. For example, a plant is an irregularly shaped object. Segmenting the plant into the small segments allows for easier calculation.

In one embodiment, the term “surface area of the plant” refers to the parts of the plant that are exposed, e.g., parts of the plant above the ground, leaves that are opened, etc. In one embodiment, the surface area of a plant is increased, e.g., opening a leaf to expose the area underneath the leaf, opening a structure such as a cone, etc.

In one embodiment, the plants disclosed herein comprise lupulin glands on about 25-100% of the surface area of the plant.

In one embodiment, the plants disclosed herein comprise lupulin glands on about 50-100% of the surface area of the plant.

In one embodiment, the plants disclosed herein comprise lupulin glands on about 70-100% of the surface area of the plant.

In one embodiment, the plants disclosed herein comprise lupulin glands on about 90-100% of the surface area of the plant.

In one embodiment, the plants disclosed herein comprise lupulin glands on about 25-50% of the surface area of the plant.

In one embodiment, the plants disclosed herein comprise lupulin glands on about 50-75% of the surface area of the plant.

In one embodiment, a vector is suspended in a buffer solution. In one embodiment, a plasmid is suspended in a buffer solution. In one embodiment, a bacteria is suspended in a buffer solution. In one embodiment, a purified transformed bacteria is suspended in a buffer solution.

As used herein, the term “suspended” refers to the settlement of particles in a medium, e.g., a liquid, a gel, a gas, etc. Suspension is due to the motion of the particles through a medium in response to the forces acting on them, e.g., gravity, centrifugal acceleration, chemical bonds, electromagnetism, etc.

As used herein, the term “buffer solution” refers to an aqueous solution consisting of a mixture of a weak acid and its conjugate base, or vice versa. In one embodiment, a buffer solution maintains the pH of a solution consistent and prevents drastic changes when new compounds or materials are added. In one embodiment, a buffer solution prevents the degradation of a transformed bacteria, e.g., maintaining the structural integrity of a transformed bacteria, maintaining the chemical composition of a transformed bacteria, etc.

In one embodiment, the plants disclosed herein comprise about 20-80 mass % of secondary compounds.

In one embodiment, the plants disclosed herein comprise about 30-70 mass % of secondary compounds.

In one embodiment, the plants disclosed herein comprise about 40-65 mass % of secondary compounds.

In one embodiment, the plants disclosed herein comprise about 30-60 mass % of secondary compounds.

In one embodiment, the plants disclosed herein comprise greater that 80 mass % of secondary compounds.

As used herein, the term “mass % of secondary compounds” refers to the percentage of the plant's total mass that is comprised of secondary compounds. For example, a plant having a total mass of 1 kilogram, comprising 500 total grams of secondary compounds, would have 50 mass % of secondary compounds.

Disclosed herein is a new plant of genus humulus comprising a cDNA sequence corresponding to lupulin gland induction in a plant not of genus humulus.

As used herein, the term “plant not of the genus humulus” means a plant not categorized into the genus humulus within the accepted biological taxonomical system. For example, a “plant not of the genus humulus” includes a plant of the genus Arabidopsis.

Disclosed herein is a method of producing secondary compounds in a of genus humulus, comprising inducing lupulin gland development in a plant of genus humulus. In some embodiments, the secondary compounds are chosen from alpha acids, beta acids, essential oils, flavonoids, or terpenes.

As used herein, the term “terpene” refers to a compound belonging to a large class of compounds often biosynthesized from 5-carbon isoprene units. In one embodiment, a terpene is produced by a variety of plants, e.g., conifers, cannabis, basil, etc. In one embodiment, a terpene is produced by an insect, e.g., termites or swallowtail butterflies. In one embodiment, a terpene is a volatile compound. In one embodiment, a terpene produces an odor. In one embodiment, a terpene is a major component of a natural resin, e.g., turpentine produced from resin. In one embodiment, a terpene is derived biosynthetically from units of isoprene, which has the molecular formula C₅H₈. In one embodiment, the molecular formula of terpenes are multiples of (C₅H₈)ₙ, where n is the number of linked isoprene units.

Within the context of this disclosure when a terpene is modified chemically, such as by oxidation or rearrangement of the carbon skeleton, the resulting compound is referred to as a terpenoid. In the relevant arts, terpenoids are sometimes referred to as isoprenoids.

In one embodiment, a terpene is the primary constituent or constituents of an essential oil from a plant and/or flower. Essential oils are used widely as fragrances in perfumery, medicine, and alternative medicines, e.g., aromatherapy.

Examples of terpenes within the context of this disclosure include: 7,8-dihydroionone, Acetanisole, Acetic Acid, Acetyl Cedrene, Anethole, Anisole, Benzaldehyde, Bergamotene (α-cis-Bergamotene) (α-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (α-Cedrene Epoxide), Cedrol, Cembrene, Chlorogenic Acid, Cinnamaldehyde (α-amyl-Cinnamaldehyde), (α-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/Icosane, Elemene (β-Elemene), Estragole, Ethyl acetate, Ethyl Cinnamate, Ethyl maltol, Eucalyptol/1,8-Cineole, Eudesmol (α-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1(10), 11-diene, Guaiacol, Guaiene (α-Guaiene), Gurjunene (α-Gurjunene), Herniarin, Hexanaldehyde, Hexanoic Acid, Humulene (α-Humulene) (β-Humulene), Ionol (3-oxo-α-ionol) (β-Ionol), Ionone (α-Ionone) (β-Ionone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, α-Sinensal, (β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, α-Tocopherol, Tonka Undecanone, Undecanal, Valeraldehyde/Pentanal, Verdoxan, α-Ylangene, Umbelliferone, or Vanillin.

In one embodiment, a terpene is chosen from Alpha-Pinene, Beta-Pinene, Camphene, Beta-Myrcene, Cymene, D-Limonene, Beta-Ocimene (Trans and Cis), Linalool, Geraniol, Alpha-Ylangene, Alpha-Copaene, Trans-Alpha-Bermotene, Gamma-Muurolene, Gamma-Selinene, Beta-Selinene, Alpha-Murrolene, Alpha-Selinene, Gamma-Cadienene, Calamenene, Delta-Cadinene, Humolene Oxide, Limonene, Terpinolene, p-Cymene, Alpha-Humulene, Beta-Caryophyllene, Trans-Beta-Farnesene, 1,4-Cineole, Beta-Citronellol, Nerol, Alpha-Terpineol, Nerolidol (Trans and Cis), Beta-Farnesene, Alpha-Terpinene, Gamma-Terpinene, Bisabolol, Iso-Pulegol, Eucalyptol, Ocimene, Guaiol, Alpha-Phellandrene, Beta-Phellandrene, Sabinene, Germacrene B, Germacrene D, Beta-Humulene, or Gamma-Caryophyllene (Iso-Caryophyllene).

In one embodiment, a terpene is chosen from Beta-Myrcene, Alpha-Humulene, and Beta-Caryophyllene.

As used herein, the term “alpha acid” refers to a class of compounds with the following structural formula:

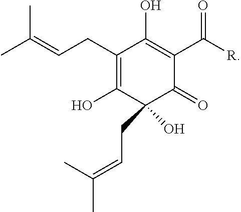

The R group is chosen from a number of substituents, e.g., methane, ethane, propane, butane (isobutane and n-butane), alcohols, ketones, etc. Alpha acids are also referred to as humulones. Alpha acids are known to readily undergo isomerization to form iso-alpha acids through heat. Iso-alpha acids contribute to the bitter taste of beer. Iso-alpha acids are also known as isohumulones. In one embodiment, alpha acids are converted to iso-alpha acids by boiling hop flowers in water. In one embodiment, alpha acids are not soluble in wort but iso-alpha acids are soluble. In one embodiment, an alpha acid is humulone. In one embodiment, an alpha acid is adhumulone. In one embodiment, an alpha acid is cohumulone. In one embodiment, an alpha acid is posthumulone. In one embodiment, an alpha acid is prehumulone.

As used herein, the term “beta acid” refers to a class of compounds with the following structural formula:

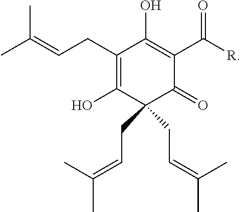

The R group is chosen from a number of substituents, e.g., methane, ethane, propane, butane (isobutane and n-butane), alcohols, ketones, etc. Beta acids are also referred to as lupulones. Beta acids differ from alpha acids in that beta acids do not readily undergo isomerization. Beta acids are also known to readily undergo oxidation, which often produces a bitter taste. In one embodiment, a beta acid is lupulone. In one embodiment, a beta acid is colupulone. In one embodiment, a beta acid is adlupulone.

As used herein, the term “essential oil” refers to a volatile aroma compound or compounds from a plant. An essential oil comprises the well-known scent or aroma of a plant. In one embodiment, an essential oil comprises a terpene. In one embodiment, a terpene is myrcene. In one embodiment, a terpene is humulene. In one embodiment, a terpene is caryophyllene.

In some embodiments, flavonoids are generally considered to be 15 carbon structures with two phenyl rings and a heterocyclic ring. There is potential overlap in which a flavonoid is also considered a terpene. However, not all terpenes could be considered flavonoids.

In one embodiment, a flavonoid is xanthohumol. In one embodiment, a flavonoid is isoxanthohumol. In one embodiment, a flavonoid is 8-prenylnaringenin.

In one embodiment, the methods disclosed herein comprise harvesting at least one alpha acid, beta acid, essential oil, flavonoid, or at least one terpene during the vegetative growth cycle of the plant of the genus humulus. In one embodiment, harvesting comprises drying the plant of genus humulus and grinding the dried plant material. In one embodiment, harvesting comprises collecting lupulin glands from a plant of genus humulus.

As used herein, the phrase “harvesting . . . during the vegetative growth cycle” means collecting secondary compounds while the plant is within the vegetative stage of growth as opposed to waiting until the plant flowers.

In one embodiment, the method of harvesting at least one alpha acid, beta acid, essential oil, flavonoid, or terpene during the vegetative growth cycle of the plant of the genus humulus includes modifying genetic material of the plant of the genus humulus.

In one embodiment, the methods disclosed herein comprise:
Constructing an endonuclease enzyme targeting a nucleic acid sequence;
Introducing the endonuclease enzyme into a genome of a plant of genus humulus; and
Manipulating expression of genes within the genome.

In one embodiment, the endonuclease enzyme is a CRISPR/Cas9 system.

As used herein, the term “CRISPR” refers to an acronym meaning Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool that complexes to and works with a compatible endonuclease protein such as Cas9 or others.

As used herein, the term “Cas” refers to a CRISPR associated protein acting as an enzyme cutting the genome at specific sequences. Cas9 refers to a specific protein within an ever-expanding group of proteins, which all share the common benefit that they are capable of being guided by a guide RNA molecule. Aside from Cas9, other suitable proteins include Cpf1. In one embodiment, a C2c2RNA sequences made from CRISPR directs a Cas9 enzyme to cut certain sequences found in a genome of an organism. Other classes of Cas are also possible.

In one embodiment, the CRISPR/Cas9 system cleaves one or two chromosomal strands at known Cas9 protein domains.

In one embodiment, one of the two chromosomal strands is mutated.

In one embodiment, two of the two chromosomal strands are mutated.

As used herein, the term “chromosomal strand” refers to a strand of DNA within a chromosome. When the CRISPR/Cas9 system cleaves the chromosomal strands, the strands are cut leaving the possibility of one or two strands being mutated, either the template strand or coding strand.

As used herein, the term “template strand of DNA” refers to the sequence of DNA used for synthesizing mRNA. In one embodiment, a template strand of DNA is used to create a complementary DNA sequence, sometimes referred to as cDNA.

As used herein, the term “coding strand of DNA” refers to the sequence of DNA that corresponds to the codons, which are ultimately translated into proteins. In most cases, either strand of chromosomal DNA could be the “coding strand” or the “template strand”. The inherent structure of the DNA strand could be relevant in determining which strand is the coding strand and which strand is the template strand.

As used herein, the term “mutated” means changing a nucleotide or nucleotides in a genetic sequence causing a change in the naturally occurring genetic sequence. The change in genetic sequence in turn affects the intended function of a protein or enzyme made from the genetic sequence. Alternative methods of DNA cutting or mutation include TALENS, zinc finger nucleases, etc.

In one embodiment, the CRISPR/Cas9 system cleaves both strands inducing non-homologous end joining (NHEJ) and then an insertion/deletion (INDEL) causing the protein to mutate and become nonfunctional. In one embodiment, the non-functionality results from a nonsense mutation that causes a premature stop codon.

In one embodiment, the CRISPR/Cas9 system cleaves both strands causing homology directed repair (HDR) to occur. In one embodiment, a donor DNA strand is inserted into the space between the cleaved strands preventing random mutation. In one embodiment, the donor DNA strand is a DNA sequence coding for aromatic prenyltransferase. In one embodiment, the donor strand is a noncoding DNA sequence.

In one embodiment, the methods disclosed herein comprise inserting a donor strand of DNA into the genome of the plant of genus humulus.

In one embodiment, the donor strand of DNA is a gene sequence coding for aromatic prenyltransferase.

As used herein, the term “donor strand of DNA” refers to genetic material inserted into the genome, a strand of DNA, a gene, etc. The donor strand of DNA may be coding or noncoding. In one embodiment, a donor strand of DNA is inserted into the cut sites of DNA to prevent mutations from occurring from DNA repair. In one embodiment, a donor strand of DNA is inserted into the cut sites of DNA to induce mutation.

In one embodiment, the methods disclosed herein comprise a utilizing a RNA guide.

As used herein, the term “RNA guide” refers to a strand of RNA recognizing a specific sequence of genetic material and directing where the endonuclease enzyme to cut.

Within the context of this disclosure, other examples of endonuclease enzymes include SpCas9 from Streptococcus pyogenes and others. Additionally, SpCas9 have differing Protospacer Adjacent Motif (PAM) sequences from NGG, which may offer other advantages. In one example, a SpCas9 has a smaller coding sequence.

Other examples of proteins compatible with CRISPRs or RNA guides include Cpf1, which can be used for cutting DNA strands with overhanging ends instead of blunt ends, or C2c2 for cutting RNA with an RNA guide.

As used herein, the term “PAM” refers to a short DNA base pair sequence immediately following the DNA sequence targeted by an endonuclease enzyme. In one embodiment, the endonuclease enzyme is a CRISPR/Cas9 system.

As used herein, the term “NGG” means a 3 nucleobase sequence with a variable followed by two Gs. “N” means any nucleobase while “G” means guanine nucleobases. In one embodiment, the methods disclosed herein comprise an endonuclease enzyme and an RNA guide.

In one embodiment, the methods disclosed herein comprise introducing a Cas9 enzyme and guide RNA expression cassette into the genome.

In one embodiment, the methods disclosed herein comprise a guide RNA transcribed in vitro.

In one embodiment, the methods disclosed herein comprise a guide RNA transcribed in vivo.

Within the context of this disclosure, cleaving a sequence of a functional gene causes a mutation, sequence change, rearrangement, etc., destroying or changing the functionality of an enzyme or protein expressed from the gene.

As used herein, the term “interfering with expression” means hindering the ability of the genome to express functional gene products. In one embodiment, interfering with expression is accomplished via knockdown. In one embodiment, interfering with expression is accomplished via knockout.

As used herein, the term “knockout” refers to the process of cutting out genes coding for enzymes, proteins, molecules, or compounds.

As used herein, the term “knockdown” refers to the process of interfering with the transcription, post transcription, pre-translation, translation, etc., of genetic information into enzymes, proteins, molecules, or compounds.

In one embodiment, the methods disclosed herein comprise interfering with expression via RNAi.

In one embodiment, the methods disclosed herein comprise interfering with the expression of a lupulin gland gene.

As used herein, the term “RNAi” refers to RNA interference. RNAi is a method of gene silencing by interfering with messenger RNA, aka mRNA. In one embodiment, miRNA (microRNA) and siRNA (small interfering RNA) molecules bind to specific sequences of mRNA, degrading the mRNA, and preventing translation of certain proteins or enzymes. In one embodiment, RNA induced silencing complexes (RISC) comprise an argonaute protein (a type of endonuclease enzyme) cleaving the targeted mRNA.

In one embodiment, the methods disclosed herein comprise introducing additional copies DNA native to the plant of the genus humulus.

As used herein, the term “introducing additional copies” means adding more genes coding for particular copies of enzymes within the plant of genus humulus.

In one embodiment, “modifying genetic material of the plant of the genus humulus” includes independently overexpressing one or more single genes inducing lupulin gland development. In one embodiment, one or more genes are chosen from available literature, and isolated from the closest relative with published sequence data.

In one embodiment, isolated DNA was inserted into an expression cassette. Overexpression of mRNA was accomplished via a CaMV 35S promoter sequence. Robust protein expression was accomplished with AtADH 5′ UTR and HSP 3′ UTR sequences. This expression cassette was inserted into the target humulus genera plant genome using a binary vector Agrobacterium mediated system. Small scale transgenesis was accomplished at a local scale with syringe infiltration, and in the whole plant via vacuum infiltration.

In one embodiment, the methods disclosed herein comprise underexpressing genetic information, e.g., a DNA sequence, a chromosome, a gene, etc. In one embodiment, underexpressing genetic information comprises removing genetic information. In one embodiment, underexpressing genetic information comprises reducing the number of functional proteins created from genetic material.

In some embodiments, the method of harvesting at least one alpha acid, beta acid, essential oil, flavonoid, or terpene during the vegetative growth cycle of the plant of the genus humulus includes introducing non-native DNA to the plant of the genus humulus.

In some embodiments, the method of harvesting at least one alpha acid, beta acid, essential oil, flavonoid, or terpene during the vegetative growth cycle of the plant of the genus humulus includes introducing additional copies DNA native to the plant of the genus humulus.

In some embodiments, the method of harvesting at least one alpha acid, beta acid, essential oil, flavonoid, or terpene during the vegetative growth cycle of the plant of the genus humulus includes overexpressing at least one lupulin gland gene.

As used herein, the term “lupulin gland gene” refers to genetic material responsible for, directly and indirectly, a lupulin gland or lupulin glands developing on a plant of genus humulus. In one embodiment, a lupulin gland gene brings about the induction of a lupulin gland on the surface area of a plant of genus humulus. In one embodiment, a lupulin gland gene indirectly hinders the development of a lupulin gland, e.g., a gene coding for a trichome expressor.

In one embodiment, the methods disclosed herein comprise over expressing at least one lupulin gland gene and interfering with expression of a lupulin gland gene.

In some embodiments, at least one lupulin gland gene is chosen from a bHLH Basic Helix Loop Helix transcription factor, a R2R3 MYB transcription factor, a R3 MYB transcription factor, a WD40 repeat transcription factor/ protein, or a homeodomain transcription factor. Notably, the preceding list of lupulin gland genes is exemplary and nonexclusive due to the complex and non-standard naming conventions. For example, the above-described homeodomain transcription factors may also be referred to as WRKY transcription factors, due to their W-box binding properties.

In some embodiments, the method of harvesting at least one alpha acid, beta acid, essential oil, flavonoid, or terpene during the vegetative growth cycle of the plant of the genus humulus includes infecting cells of the plant of genus humulus with a transformed bacteria.

In some embodiments, the method disclosed herein includes infecting cells of a plant of genus humulus with a transformed bacteria via syringe infiltration.

In one embodiment, “syringe infiltration” was accomplished as follows: a 10 ml or 50 ml syringe, without a needle, was loaded with a bacteria solution. The syringe tip was placed flat against the underside of the leaf. A gloved finger was placed against the top of the leaf to apply pressure. The plunger was slightly depressed, allowing the fluid to travel through the open stomata into the intercellular space in the leaf, where the bacteria infects the cells and subsequently inserts the expression cassette with the gene of interest. This procedure was carried out when the plants were under bright lighting to ensure active transpiration and open stomata. Plants were grown and assessed for expression at the injection site. Localized transgenesis was observed.

In some embodiments, the method disclosed herein includes infecting cells of a plant of genus humulus with a transformed bacteria via vacuum infiltration.

In one embodiment, “vacuum infiltration” was accomplished as follows: Small rooted clones were suspended upside down in a bath of bacteria solution; all leaves, stems and growth tips were submerged and the roots left exposed; the bath was situated in a vacuum chamber and a vacuum was applied to the roots, thereby pulling the solution into the leaves through the open stomata and the rest of the plant via the vascular system. This procedure resulted in mosaic expression of the transgene in the whole plant. The plant was then grown and sub-cloned using traditional plant cloning and/or plant tissue culture.

In one embodiment, the sub cloning step comprises using an antibiotic or other selective regime such as an herbicide, broadly toxic antibiotic, chemotype screening, monitoring for expression of anther transgene, identification of visually different phenotype, etc. The clone was usable in a normal humulus cultivation scenario, while expressing the transgene and its resulting phenotype.

In one embodiment, the method of harvesting at least one alpha acid, beta acid, essential oil, flavonoid, or terpene during the vegetative growth cycle of the plant of the genus humulus includes treating cells of the plant of genus humulus with DNA attached to a metal particle via biolistic particle delivery.

As used herein, the term “metal particle” refers to a piece of material typically hard, opaque, shiny, and having electrical and thermal conductivity.

As used herein, the term “biolistic particle delivery” refers to the delivery of exogenous DNA (transgenes) to cells. In one embodiment, the delivery is an elemental particle of a heavy metal coated with DNA, e.g., a plasmid comprising DNA.

In one embodiment, the methods disclosed herein comprise:

repressing post-transcriptional processing of a transcriptional repressor of lupulin gland induction; and

repressing expression of a target gene as a functional protein.

In one embodiment, the methods disclosed herein comprise:

expressing an RNA interference molecule corresponding to a lupulin gland gene; and

reducing translation of mRNA lupulin gland gene into a functional protein.

In one embodiment, the method of harvesting at least one alpha acid, beta acid, essential oil, flavonoid, or terpene during the vegetative growth cycle of the plant of the genus humulus includes repressing post-transcriptional processing of a transcriptional repressor of lupulin gland induction and/or repressing expression of a target gene as a functional protein.

As used herein, the term “repressing” refers to inhibiting, hindering, and/or down regulating expression of genetic material, e.g., a gene, a DNA sequence, etc. In one embodiment, repressing refers to inhibiting the biosynthesis of inhibitors or repressors. In one embodiment, the methods disclosed herein comprise repressing a lupulin gland gene.

As used herein, the term “expression” refers to the process by which genetic material is used to make functional gene products. In one embodiment, the methods disclosed herein are used to bolster the expression of a lupulin gland gene.

As used herein, the term “post-transcriptional processing” is the process where primary transcript RNA is converted into mature RNA. Post-transcriptional processing includes the addition of a 5′ cap, the addition of a 3′ polγ-adenylation tail, and splicing. Post-transcriptional processing is important for correcting translation of the genome because the initial precursor mRNA produced during transcription contains both exons (coding or important sequences involved in translation) and introns (non-coding sequences).

As used herein, “transcriptional repressor” refers to a protein binding to a specific site on a DNA strand and preventing the transcription of nearby genes. Most repressors inhibit the initiation of transcription.

As used herein, the term “target gene” refers to a specific gene desired to be translated into a functional gene product, e.g., a functional protein.

In some embodiments, the method of harvesting at least one alpha acid, beta acid, essential oil, flavonoid, or terpene during the vegetative growth cycle of the plant of the genus humulus includes expressing an RNA interference molecule corresponding to a lupulin gland induction repression gene and/or reducing translation of mRNA lupulin gland induction repression genes into a functional protein.

As used herein, the term “RNA interference molecule” refers to an RNA molecule inhibiting the translation of proteins and/or enzymes. RNA interference molecules are used to bind to specific sequences of mRNA, degrading the mRNA, and preventing translation of certain proteins or enzymes. RNA induced silencing complexes (RISC) comprise an argonaute protein (a type of endonuclease enzyme), which cleaves the targeted mRNA.

As used herein, the term “reducing translation” refers to decreasing and/or interfering with the amount of proteins made. In one embodiment, reducing translation is accomplished by destroying mRNA molecules. In one embodiment, reducing translation is accomplished by removing the start codon.

As used herein, the term “functional protein” refers to a molecule or molecules composed of amino acids performing an action within an organism. In one embodiment, a functional protein is hemoglobin. In one embodiment, a functional protein is an enzyme.

In some embodiments, the method of harvesting at least one alpha acid, beta acid, essential oil, flavonoid, or terpene during the vegetative growth cycle of the plant of the genus humulus includes modifying expression (in a plant of genus humulus) of a gene chosen from a bHLH Basic Helix Loop Helix transcription factor, a R2R3 MYB transcription factor, R3 MYB transcription factor, a WD40 repeat transcription factor/ protein, or a homeodomain transcription factor.

In some embodiments, the method of harvesting at least one alpha acid, beta acid, essential oil, flavonoid, or terpene during the vegetative growth cycle of the plant of the genus humulus includes physically damaging the DNA corresponding to a transcriptional repressor of lupulin gland induction.

As used herein, the term “physically damaging the DNA” refers to destroying, changing, and/or manipulating a DNA sequence such that the DNA sequence no longer is a viable source of genetic information. In one embodiment, physically damaging the DNA is done through mutation. In one embodiment, physically damaging the DNA is cleaving a sequence.

In one embodiment, the methods disclosed herein comprise physically altering the DNA of the plant of genus humulus.

Although the present invention herein has been described with reference to various exemplary embodiments, it is to be understood that these embodiments are merely illustrative of the principles and applications of the present invention. Those having skill in the art would recognize that various modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

Finally, it is noted that, as used in this specification and the appended claims, the singular forms “a,” “an,” and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

What is claimed is:

1. A plant of genus humulus, comprising;
lupulin glands on non-flowering parts of the plant of genus humulus;
a vector comprising genetic information; and
wherein the plant of genus humulus comprises a surface area.

2. The plant of claim 1, wherein the vector is a plasmid comprising a cDNA sequence native to a plant of genus humulus corresponding to lupulin gland induction.

3. The plant of claim 1, wherein the vector is a purified transformed bacteria comprising a DNA sequence native to a plant of genus humulus, wherein said native DNA sequence corresponds to lupulin gland induction.

4. The plant of claim 1, comprising lupulin glands on about 25-100% of the surface area of the plant.

5. The plant of claim 4, comprising lupulin glands on about 50-100% of the surface area of the plant.

6. The plant of claim 4, comprising lupulin glands on about 70-100% of the surface area of the plant.

7. The purified transformed bacteria of claim 1, comprising a buffer solution; wherein the vector is suspended in the buffer solution.

8. The plant of claim 1, comprising 20-80 mass % of secondary compounds of the plant.

9. The plant of claim 8, comprising about 30-70 mass % of secondary compounds of the plant.

10. The plant of claim 8, comprising about 40-65 mass % of secondary compounds of the plant.

11. The plant of claim 8, comprising about 50-60 mass % of secondary compounds of the plant.

12. A plant of the genus humulus comprising cDNA fragments corresponding to lupulin gland induction in a plant not of the genus humulus.

13. The plant of claim 12, wherein the plant not of the genus humulus is a plant of genus Arabidopsis.

14. A method of producing secondary compounds, comprising inducing lupulin gland development in a plant of genus humulus.

15. The method of claim 14, wherein said secondary compounds are chosen from alpha acids, beta acids, essential oils, flavonoids, or terpenes.

16. The method of claim 15, comprising harvesting at least one alpha acid or at least one terpene during the vegetative growth cycle of the plant of the genus humulus.

17. The method of claim 14, comprising modifying genetic material of the plant of the genus humulus.

18. The method of claim 14, comprising introducing non-native DNA sequence into the plant of the genus humulus.

19. The method of claim 17, comprising overexpressing at least one lupulin gland gene.

20. The method of claim 19, comprising interfering with expression of at least one lupulin gland gene.

21. The method of claim 20, wherein the at least one lupulin gland gene is chosen from a bHLH Basic Helix Loop Helix transcription factor, a R2R3 MYB transcription factor, a R3 MYB transcription factor, a WD40 repeat transcription factor/ protein, or a homeodomain transcription factor.
